(19) United States
(12) Patent Application Publication
(10) Pub. No.: US 2019/0223585 A1
(43) Pub. Date: Jul. 25, 2019

(54) BRUSH STERILIZATION UNIT

(71) Applicant: Teleo Group, LLC, Anaheim, CA (US)

(72) Inventors: Sean Wayne Wigand, Lake Forest, CA (US); Joshua John Kompa, Foothill Ranch, CA (US); Patrick Michael Curtis, Mission Viejo, CA (US)

(21) Appl. No.: 15/877,292

(22) Filed: Jan. 22, 2018

(51) Int. Cl. A46B 17/06; A61L 2/10

(52) U.S. Cl. CPC A46B 17/065; A61L 2/10; A61L 2/24; A61L 2202/14; A61L 2202/17

(57) ABSTRACT

A sterilizing unit for a brush simultaneously moves an ultraviolet light source under a brush head to kill bacteria on the bristles with UV light, and also separates the bristles from each other to remove dirt and debris from between the bristles.

### BACKGROUND

### Field

The present invention relates to a brush sterilization unit, and more particularly to a UV light sterilization unit for cosmetic brushes.

### Description of the Related Art

Cosmetic brushes are commonly used, for example, to apply makeup. However, the bristles in the brush head can retain old makeup, dirt and debris, bacteria, oils and dead skin cells. Therefore cosmetic brushes must regularly be cleaned to remove such debris and bacteria, which can aid in preventing skin reactions, such as acne, caused by the use of cosmetic brushes that are not adequately cleaned.

### SUMMARY

There is a need for an improved system to clean brushes, such as cosmetic brushes. In one aspect of the invention, a sterilization unit is provided that simultaneously sterilizes a brush head and kills bacterial trapped between the bristles and removes dirt and debris trapped in between the bristles.

In accordance with another aspect, a sterilizing unit is provided that simultaneously applies ultraviolet light to the bristles of a brush to kill bacteria and separates the bristles from each other to remove dirt and debris trapped between the bristles.

In accordance with another aspect, a sterilizing unit is provided that simultaneously moves one or more ultraviolet light sources under a brush head so that the one or more ultraviolet light sources separate bristles from each other, kills bacteria on the bristles with ultraviolet light, and knocks dirt and debris from the bristles.

In accordance with another aspect, a sterilization unit for a brush is provided. The unit comprises a base having a proximal side and a distal side and defining a housing. The unit also comprises a light source movably disposed on the proximal side of the base, the light source configured to move on at least a portion of the proximal side of the base. The unit also comprises control circuitry disposed in the housing and configured to operate the light source. Movement of the light source simultaneously separates bristles of a brush disposed over the base from each other to thereby remove debris trapped between the bristles and applies light onto the bristles and between the bristles to sterilize them.

In accordance with another aspect, a sterilization unit for a brush is provided. The unit comprises a base having a proximal side and a distal side and defining a housing. The unit also comprises at least one ultraviolet light source movably disposed on the proximal side of the base, the at least one ultraviolet light source configured to move on at least a portion of the proximal side of the base. The unit also comprises control circuitry disposed in the housing and configured to operate the at least one ultraviolet light source. The unit also comprises means for effecting movement of the at least one ultraviolet light source on the proximal side of the base so that the at least one ultraviolet light source moves underneath a brush supported on the base to simultaneously separate bristles of the brush from each other to thereby remove debris trapped between the bristles and also apply ultraviolet light onto the bristles and between the bristles to sterilize them.

The sterilization unit may be arranged such that the means for effecting movement of the at least one ultraviolet light source includes means for rotating the at least the one ultraviolet light source.

The sterilization unit may be arranged such that the means for effecting movement of the at least one ultraviolet light source includes means for translating the at least one ultraviolet light source.

In accordance with another aspect, a sterilization unit for a brush is provided. The unit comprises a base having a proximal side and a distal side and defining a housing. The unit also comprises a lid configured to couple to the base. The lid is movable between an open position relative to the base where at least a portion of a distal end of the lid is spaced apart from the base to allow access to the proximal side of the base, and a closed position relative to the base where the distal end of the lid is coupled to the base to cover the proximal side of the base. The unit also comprises a light source movably disposed on the proximal side of the base, the light source configured to move along a track defined in the proximal side of the base. The unit also comprises an electric motor disposed in the housing and operatively coupled to the light source via a lead screw, the motor operable to actuate movement of the light source along the track via the lead screw. The unit also comprises control circuitry disposed in the housing and configured to operate one or both of the electric motor and the light source. The lid is sized to receive at least a portion of a brush therein so that when the lid is in the closed position bristles of the brush are adjacent the proximal side of the base and over the light source so that the light source extends in between one or more of the bristles. Movement of the light source simultaneously separates the bristles from each other to thereby remove debris trapped between the bristles and also applies light onto the bristles and between the bristles to sterilize them.

In accordance with another aspect, a sterilization unit for a brush is provided. The unit comprises a base having a proximal side and a distal side and defining a housing. The unit also comprises a lid movably coupled to the base. The lid is movable between an open position relative to the base where at least a portion of a distal end of the lid is spaced apart from the base to allow access to the proximal side of the base, and a closed position relative to the base where the distal end of the lid is coupled to the base to cover the proximal side of the base. The unit also comprises at least one ultraviolet light source movably disposed on the proximal side of the base, the at least one ultraviolet light source configured to move along an axis on the proximal side of the base. The unit also comprises an electric motor disposed in the housing and operatively coupled to the at least one ultraviolet light source, the motor operable to actuate movement of the at least one ultraviolet light source along the axis. The unit also comprises control circuitry disposed in the housing and configured to operate one or both of the electric motor and the at least one ultraviolet light source. The lid is sized to receive at least a portion of a brush therein so that when the lid is in the closed position bristles of the brush are disposed adjacent the proximal side of the base and over the at least one ultraviolet light source so that the ultraviolet light source extends in between one or more of the bristles. Movement of the at least one ultraviolet light source simultaneously separates the bristles from each other to thereby remove debris trapped between the bristles and also applies light onto the bristles and between the bristles to sterilize them.

### BRIEF DESCRIPTION OF THE DRAWINGS

FIG. 1 is a perspective front view of a sterilization unit.

FIG. 2 is a front view of the sterilization unit of FIG. 1.

FIG. 3 is a rear view of the sterilization unit of FIG. 1.

FIG. 4 is a left side view of the sterilization unit of FIG. 1, the right side being a mirror image.

FIG. 5 is a top view of the sterilization unit of FIG. 1.

FIG. 6 is a bottom view of the sterilization unit of FIG. 1.

FIG. 7 is a cross-sectional side view of the sterilization unit of FIG. 1.

FIG. 8 is an exploded view of the sterilization unit of FIG. 1.

FIG. 9A is a perspective front view of a sterilization unit.

FIG. 9B is an exploded view of the sterilization unit in FIG. 9A.

FIG. 9C is a top perspective view of a base of the sterilization unit of FIG. 9A.

FIG. 9D is a cross-sectional view of the base of the sterilization unit of FIG. 9A.

FIG. 10A is a perspective front view of a sterilization unit.

FIG. 10B is an exploded view of the sterilization unit in FIG. 10A.

FIG. 10C is a top perspective view of a base of the sterilization unit of FIG. 10A.

FIG. 10D is a cross-sectional view of the base of the sterilization unit of FIG. 10A.

FIG. 11A is a perspective front view of a sterilization unit.

FIG. 11B is an exploded view of the sterilization unit in FIG. 11A.

FIG. 11C is a top perspective view of a base of the sterilization unit of FIG. 11A.

FIG. 11D is a cross-sectional view of the base of the sterilization unit of FIG. 11A.

FIG. 12A is a schematic view of a sterilization unit.

FIG. 12B is a schematic view of a sterilization unit.

### DETAILED DESCRIPTION

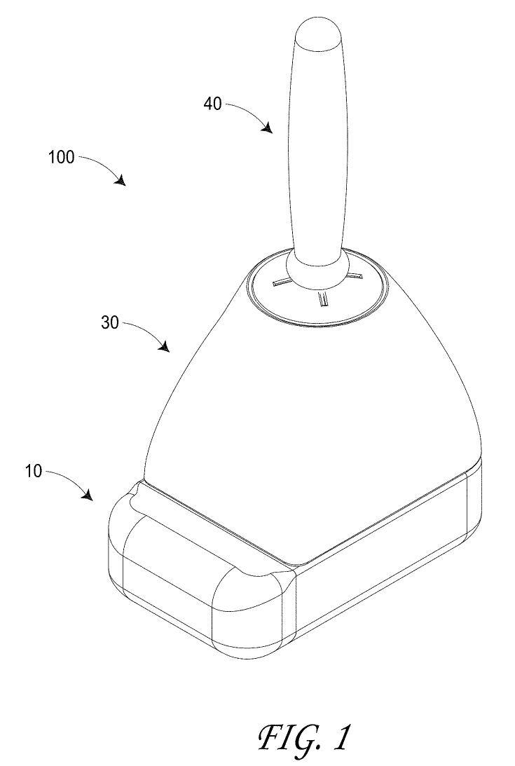

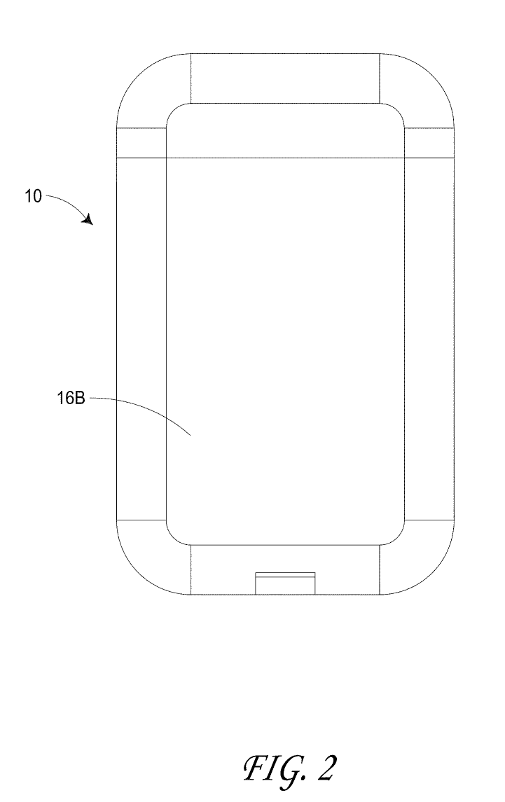

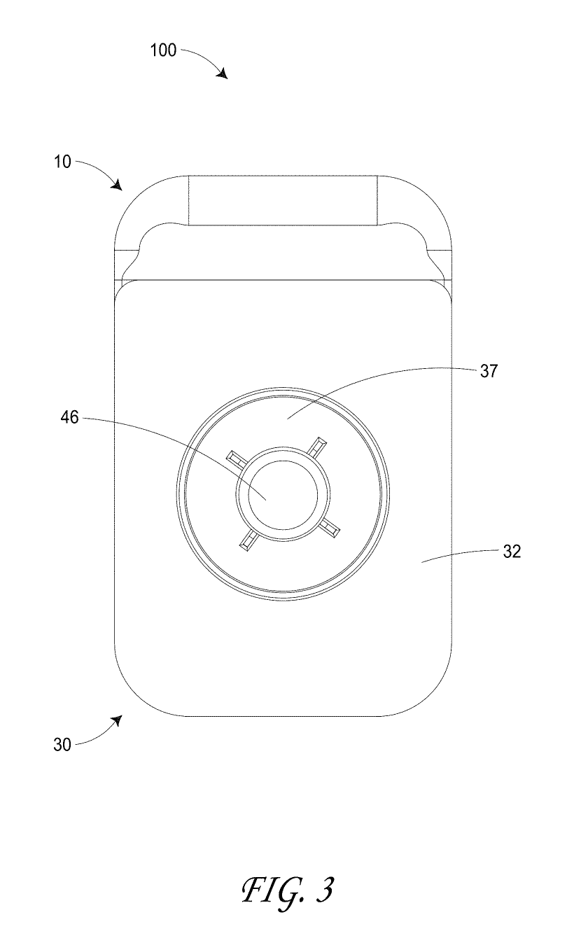

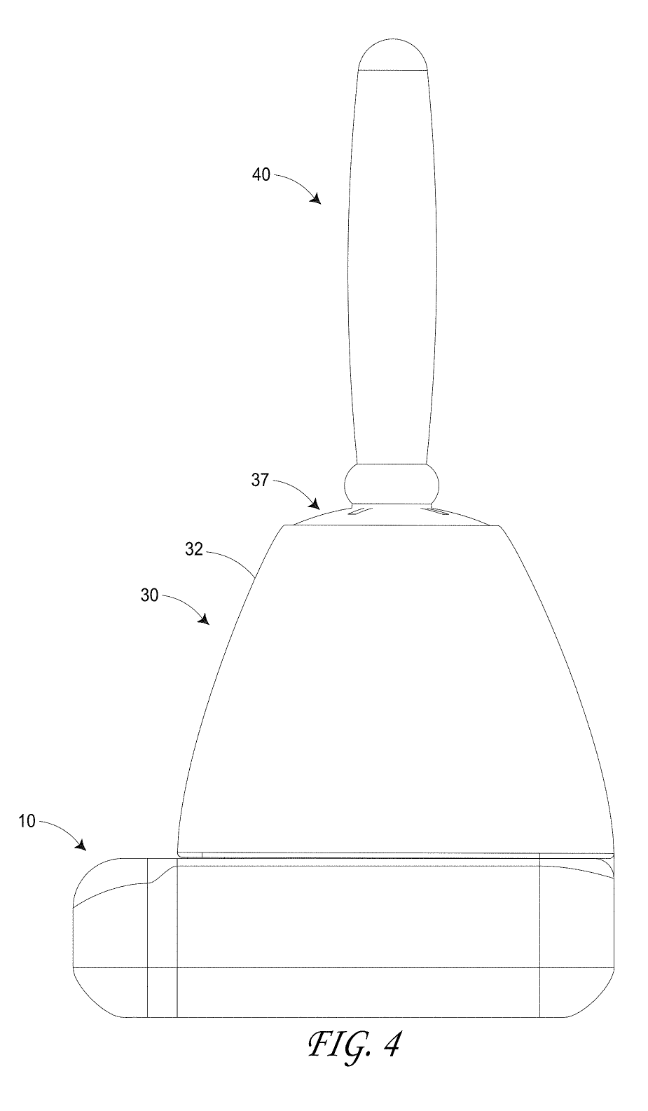

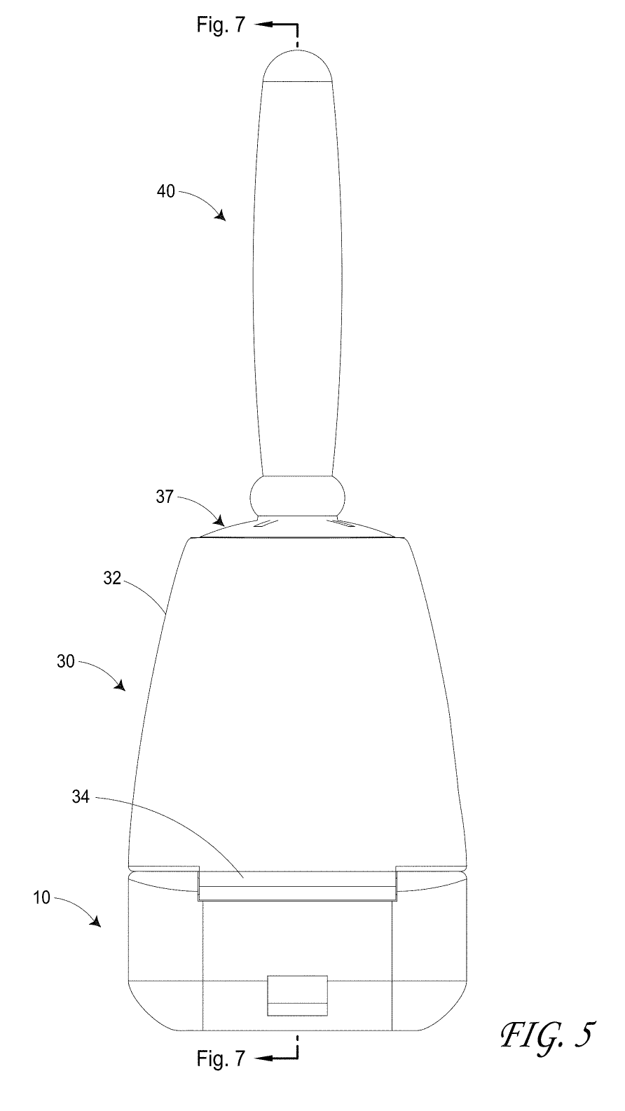

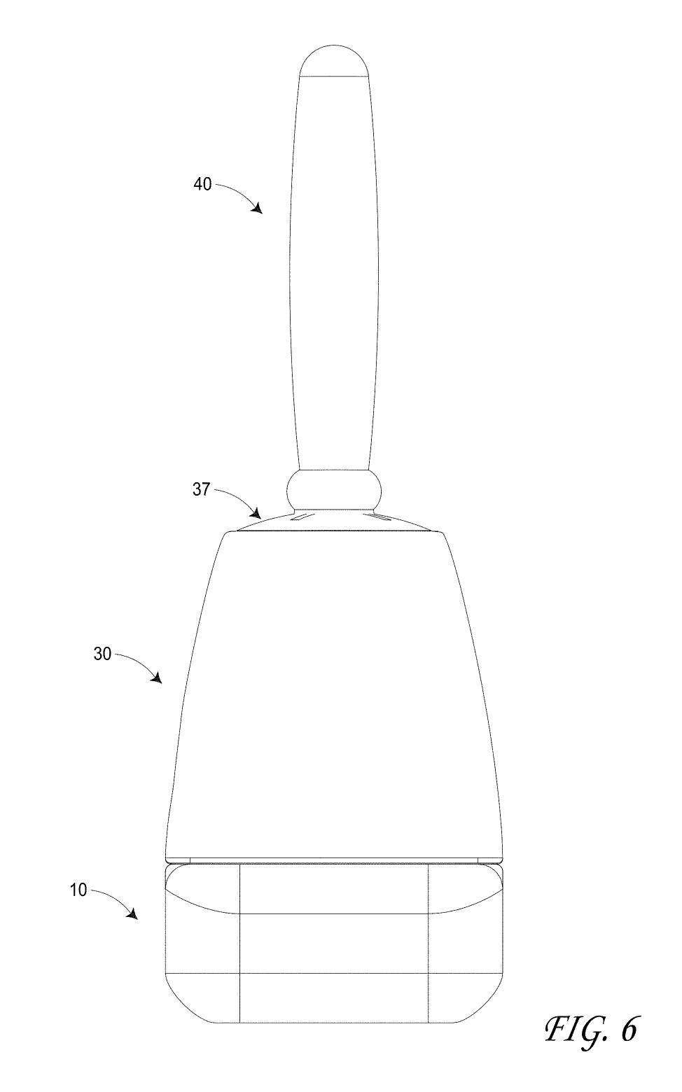

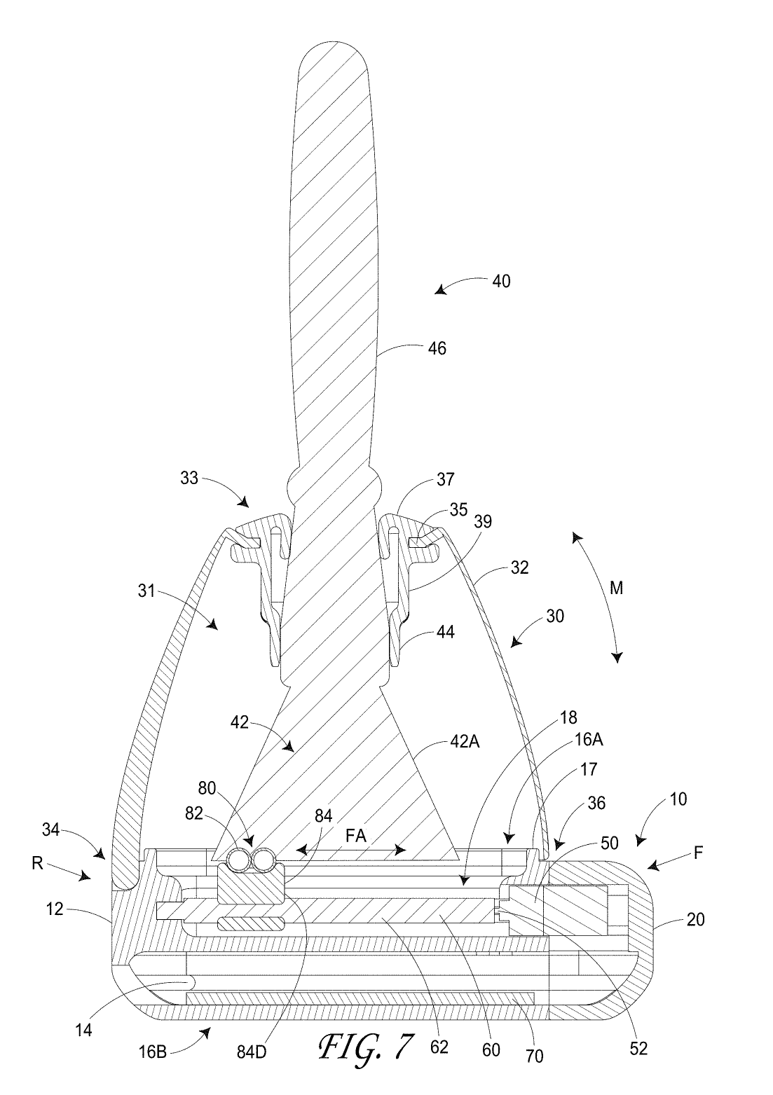

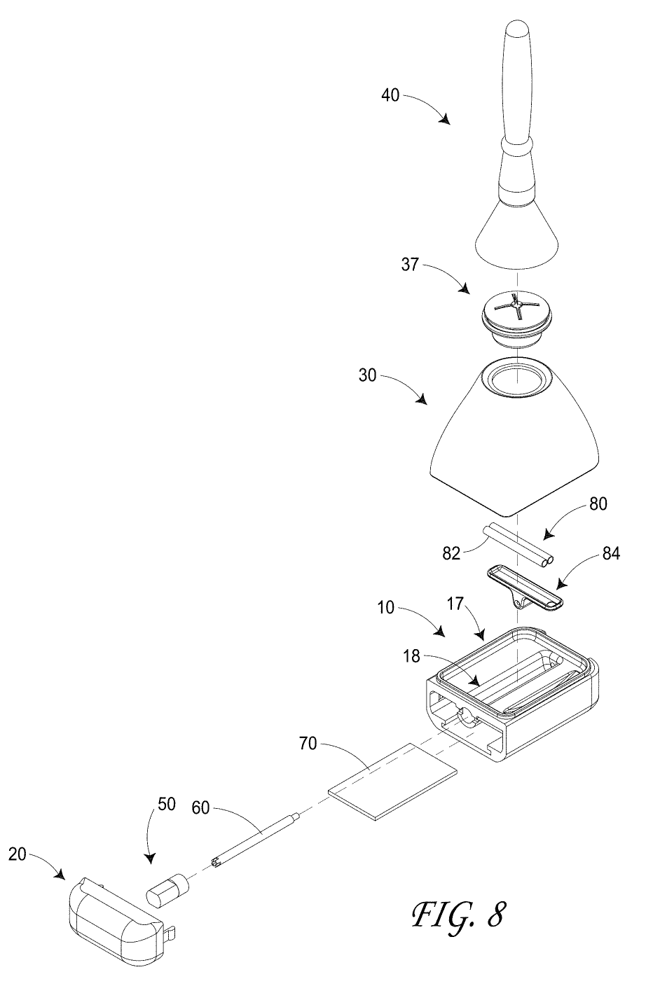

FIGS. 1-8 show a sterilization unit 100 for cleaning and/or sterilizing a brush, such as a cosmetic brush 40. The unit 100 has a base 10 and a lid or cover 30 that is movably coupled to the base 10. Optionally, a portion 34 (e.g., a rear edge portion) of the lid 30 can be pivotally coupled to the base 10, such as via an axle that extends at least partially through the base 10 and the portion 34 of the lid 30. The lid can have a second portion 36 (e.g., front edge portion) that can optionally contact a surface (e.g., a top surface) of the base 10 when the lid 30 is in a closed position (see FIG. 7) over the base 10. As shown in FIG. 7, the lid 30 can optionally pivot M about portion 34 to move the lid 30 between the closed position (shown in FIG. 7), where the second portion 36 is optionally in contact with the base 10, and an open position, where the second portion 36 is spaced apart from the base 10, thereby advantageously allowing access to an underside 31 of the lid 30. Moving the lid 30 to the open position allows for the insertion of the brush 40 into the lid 30 (e.g., prior to a cleaning operation, as further described below), and for the removal of the brush 40 from the lid 30 (e.g., once a cleaning operation has been completed). Alternatively, the lid 30 is not pivotally coupled to the base 10, and the lid 30 is instead removably attached to the base 10 via another suitable mechanism (e.g., the lid 30 is clipped to the base 10 by a separate clip component, the lid 30 removably clips directly onto a portion of the base 10, such as via a lip of the lid 30 that engages a recess in the base 10). The lid 30 can optionally be made of a transparent, translucent or opaque material. Optionally, the lid 30 is made of plastic. However, the lid 30 can be made of other suitable materials.

As shown in FIG. 7, the lid 30 holds the brush 40 substantially in place on the base during a cleaning operation. Advantageously, the lid 30 holds the brush 40 in a generally upright position (e.g., generally vertical position) relative to the base 10, so that the brush head 42 (e.g., the plurality of bristles 42a of the brush 40) are disposed facing the base 10, as further discussed below. The lid 30 has an opening 33 at a proximal end through which at least a portion of a handle 46 of the brush 40 removably extends. The opening 33 can optionally be defined by a flexible member (e.g., flexible gasket) 37 that optionally couples to the lid 30. For example, the flexible member can fasten over a flange 35 of the lid 30. The flexible member 37 can optionally have resilient arms 39 that removably hold onto a body portion 44 of the handle 40. The flexible member 37 can optionally be made of rubber, plastic, or another suitable resilient material. Alternatively, the flexible member 37 can be excluded.

The base 10 has a housing 12 with an inner cavity 14 therein. Optionally, a front portion 20 of the base 10 can be detached from the rest of the base 10 to access the cavity 14. Alternatively, the base 10 can have a bottom cover that can be detached to access the cavity 14. The base 10 can have a proximal side 16A (e.g., top side) and a distal side 16B (e.g., bottom side). Optionally, a raised lip 17 can be defined on the proximal side 16A and can extend along at least a portion of a circumference of the proximal side 16A. When the lid or cover 30 is in the closed position, the bottom edge of the lid 30 optionally extends about (e.g., surrounds) the raised lip 17. Optionally, a slot or track 18 (e.g., elongate opening) can be defined on the proximal side 16A of the base 10. The slot 18 can be an elongate opening that extends substantially linearly along an axis between a first end (e.g., a front end) F of the base 10 and a second end (e.g., a rear end) R of the base 10.

The base 10 includes a motor 50 (e.g., an electric motor) disposed in the cavity 14. The motor 50 can have an output shaft 52 that rotates upon actuation of the motor 50. The output shaft 52 can optionally couple to a rotatable member 60. The rotatable member 60 can optionally be a lead screw 60 with a plurality of threads 62, as shown in FIG. 7. Alternatively, the rotatable member 60 can be a worm gear with a plurality of gear segments. The rotatable member 60 can be rotated (e.g., clockwise, counterclockwise) by the output shaft 52 of the motor 50 upon actuation of the motor 50.

As best shown in FIG., 7, a light source 80 with one or more light members 82 (e.g., a pair of light members 82) can be attached to or otherwise supported on a carriage 84. The one or more light members 82 can optionally be ultraviolet lights. Optionally, the ultraviolet lights can operate at a wavelength of between about 300 nm and about 400 nm, such as about 355 nm. The one or more light members 82 can optionally be elongate and extend along an axis generally transverse (e.g., generally perpendicular) to a longitudinal axis of the base 10.

The carriage 84 can be movably coupled to the rotatable member 60, such that rotation of the rotatable member 60 causes a translation of the carriage 84 (and therefore a translation of the light source 80) in a fore and aft direction FA (e.g., along or within the slot or track 18). For example, the carriage 84 can have a threaded portion 84D that threadably engages the threads 62 of the lead screw 60 or worm gear portions 62 of the worm gear 60. Rotation of the rotatable member 60 in one direction (e.g., clockwise) causes translation of the carriage 84 and light source 80 in one direction (e.g., forward), and rotation of the rotatable member 60 in an opposite direction (e.g., counterclockwise) causes translation of the carriage 84 and light source 80 in an opposite direction (e.g., rearward).

The base 10 can optionally include circuitry 70, for example, on a printed circuit board. The circuitry 70 can communicate with (e.g., be electrically connected to) and control an operation of one or both of the motor 50 and light source 80. For example, the circuitry 70 can turn on the one or more light members 82 of the light source 80, and can operate the motor 50 to rotate the output shaft in a clockwise or counterclockwise direction, as well as control the speed of rotation of the output shaft. Accordingly, the circuitry 70 can advantageously operate the speed with which the carriage 84 and light source 80 translates on the base 10 and the frequency with which the carriage 84 and light source 80 move fore and aft on the base 10.

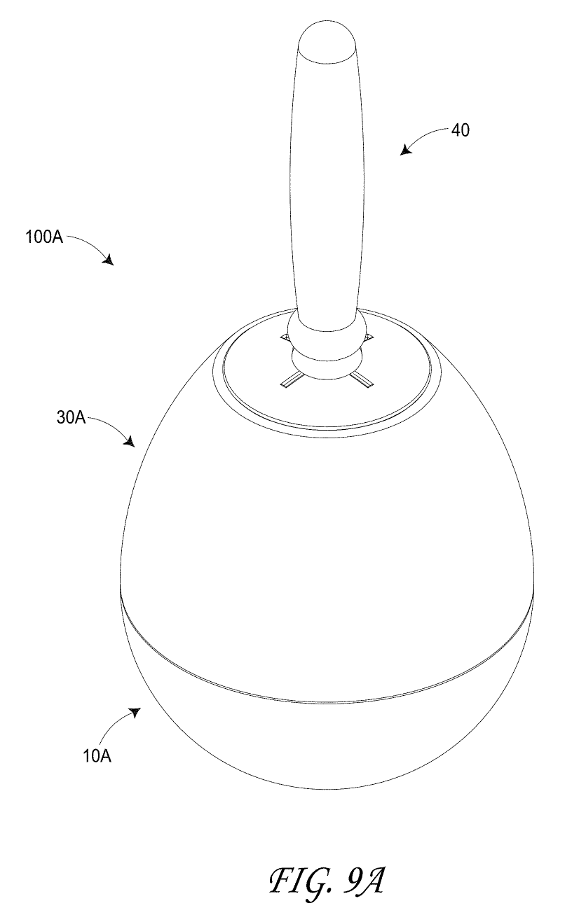

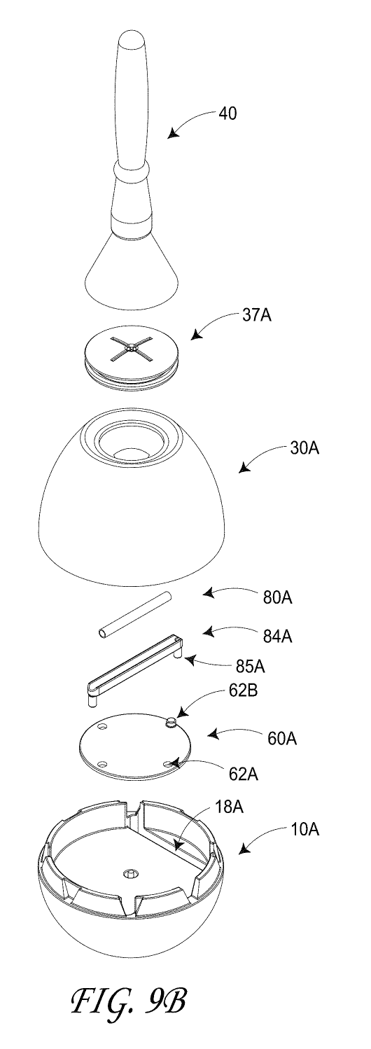

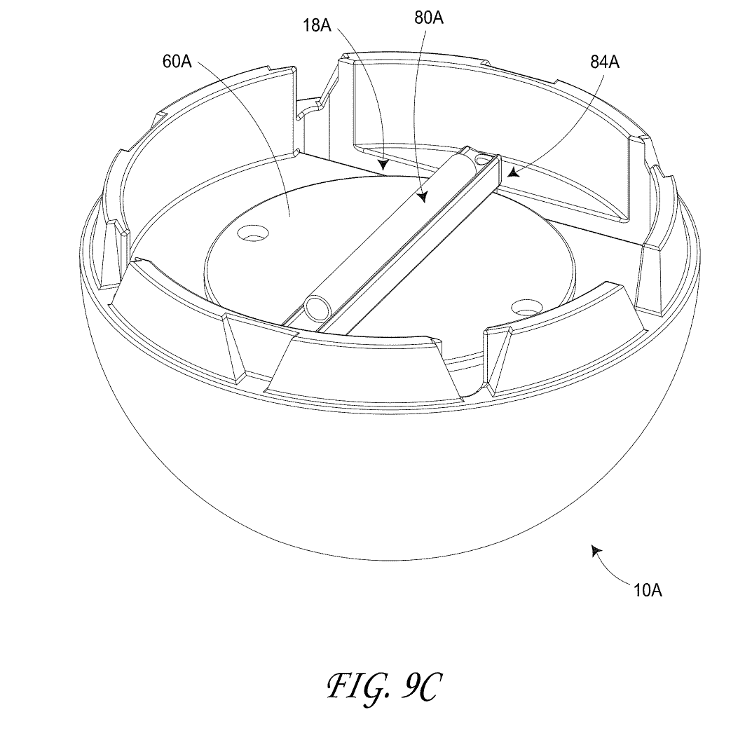

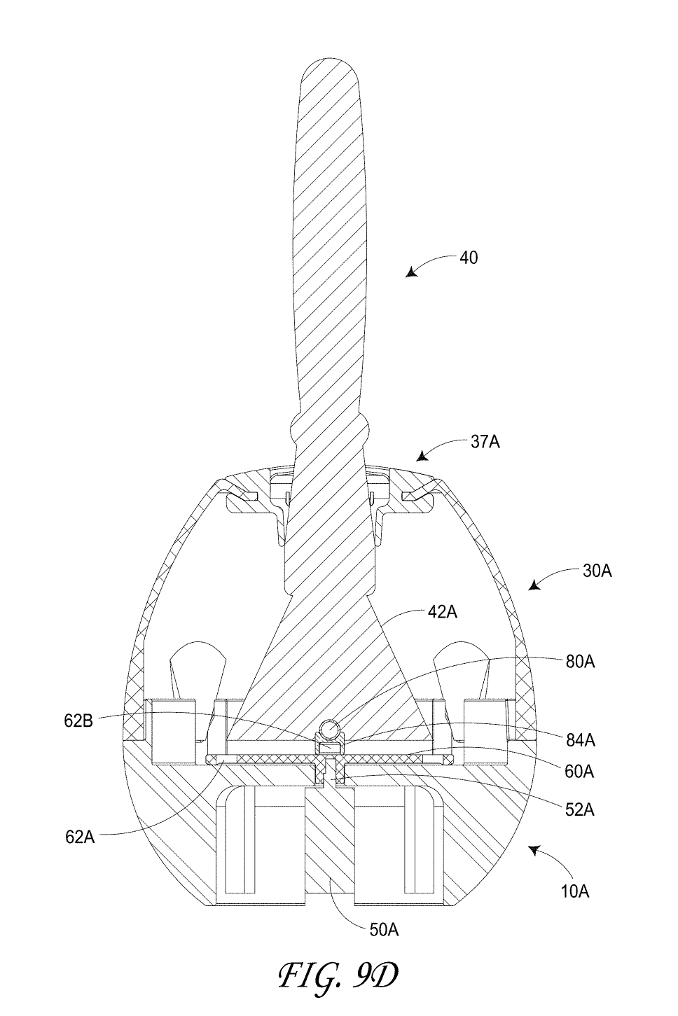

FIGS. 9A-9D show another sterilization unit 100A. The sterilization unit 100A is similar to the sterilization unit 100 in FIGS. 1-8 and includes some of the same components and features disclosed for sterilization unit 100, except as noted below. Thus, the reference numerals used to designate the various components of the sterilization unit 100A are identical to those used for identifying the corresponding components of the sterilization unit 100 in FIGS. 1-8, except that an “A” has been added to the reference numerals, and the description above of the components of the sterilization unit 100 are understood to also apply to corresponding components in the sterilization unit 100A, except as described below.

The sterilization unit 100A differs from the sterilization unit 100 in the construction of the base 10A and the manner in which the light source 80A is moved under the plurality of bristles 42A of the brush 40. The base 10A and the lid 30A optionally have a circular profile symmetrical about a central axis (e.g., axis of symmetry). The sterilization unit 100A has a motor 50A housed in the base 10A that extends along an axis that is substantially coaxial with the central axis (e.g., axis of symmetry) of the base 10A and/or the lid 30A. The motor 50A has an output shaft 52A that engages (e.g., via one or more gears) a plate 60A so that the plate 60A rotates about the axis of the output shaft 52A. The plate 60A has one or more (e.g., a plurality of) holes 62A and at least one peg 62B. The carriage 84A has a pair of pegs 85A that can extend into channels 18A in the base 10A. The peg 62B extends into a channel on an underside of the carriage 84A.

When the motor 50A is operated to rotate the output shaft 52A, rotation of the output shaft 52A rotates the plate 60A. Rotation of the plate 60A causes the peg 62B to slide along the channel of the carriage 84A, which in turn cause the carriage 84A to translate along the channels 18A via the sliding engagement of the pegs 85A of the carriage 84B with the channels 18A of the base 10A. Therefore, rotation of the output shaft 52A effects rotation of the plate 60A, which in turn effects translation of the carriage 84A (and the light source 80A disposed thereon) in a back and forth manner on the base 10A. The light sources 80A thereby translates under the bristles 42A of the brush 40 to separate the bristles 42A from each other to allow debris therebetween to fall out, and simultaneously applies light (e.g., UV light) in between the bristles 42A to sterilize them.

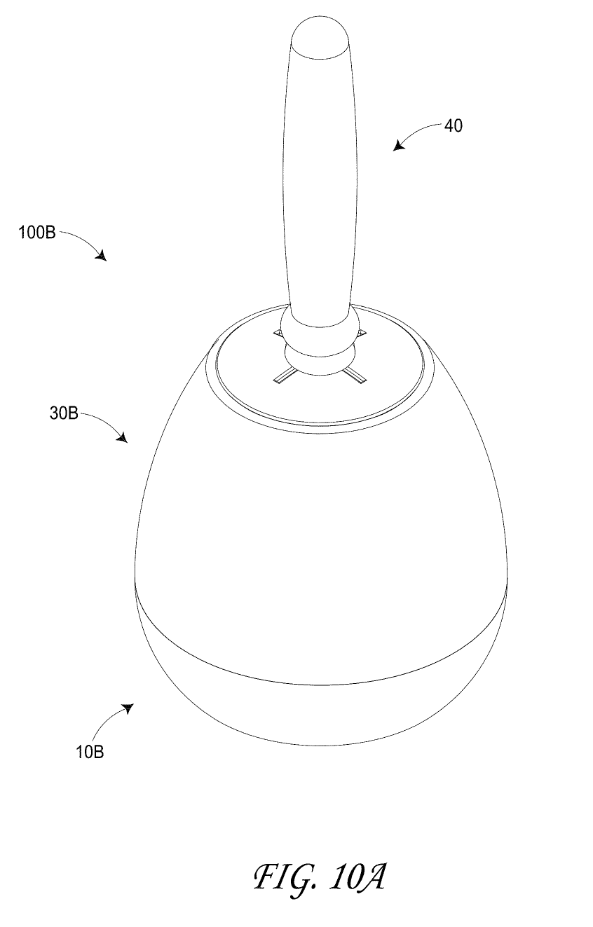

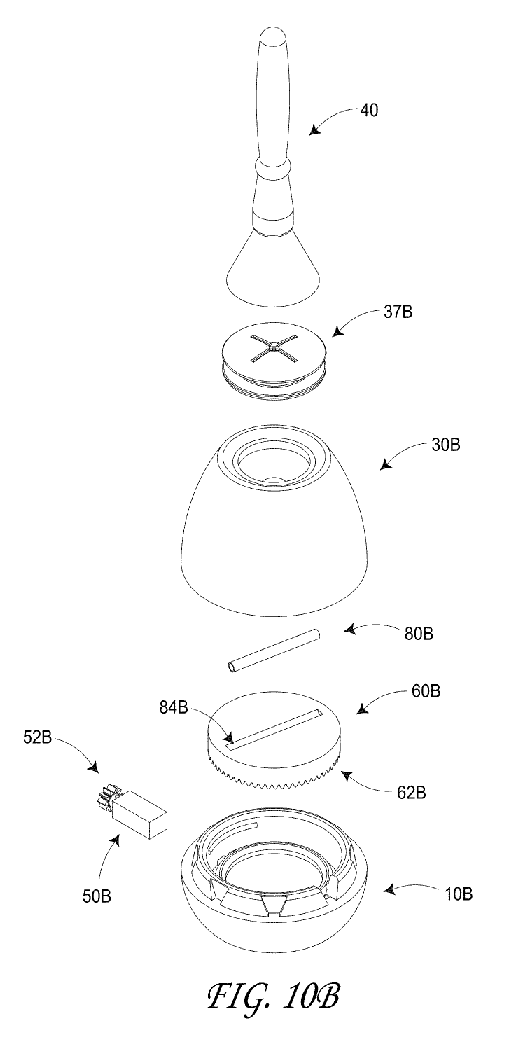

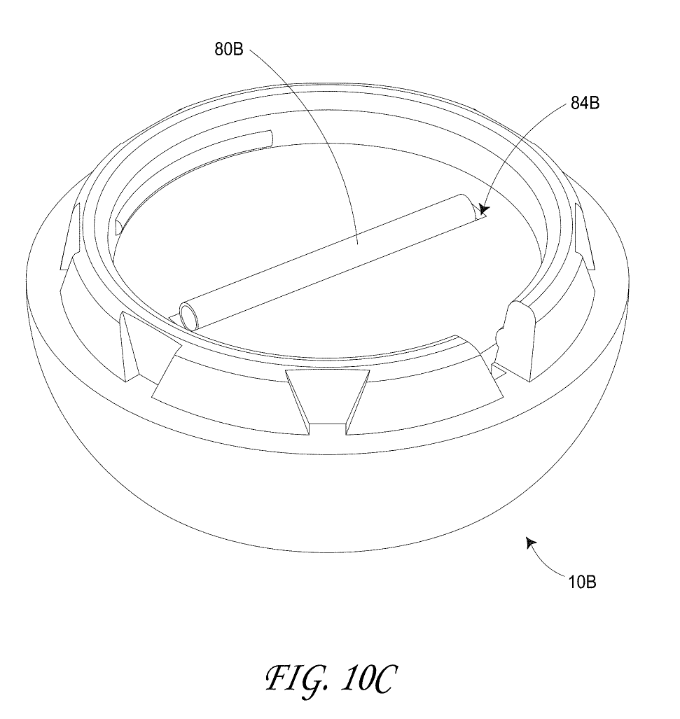

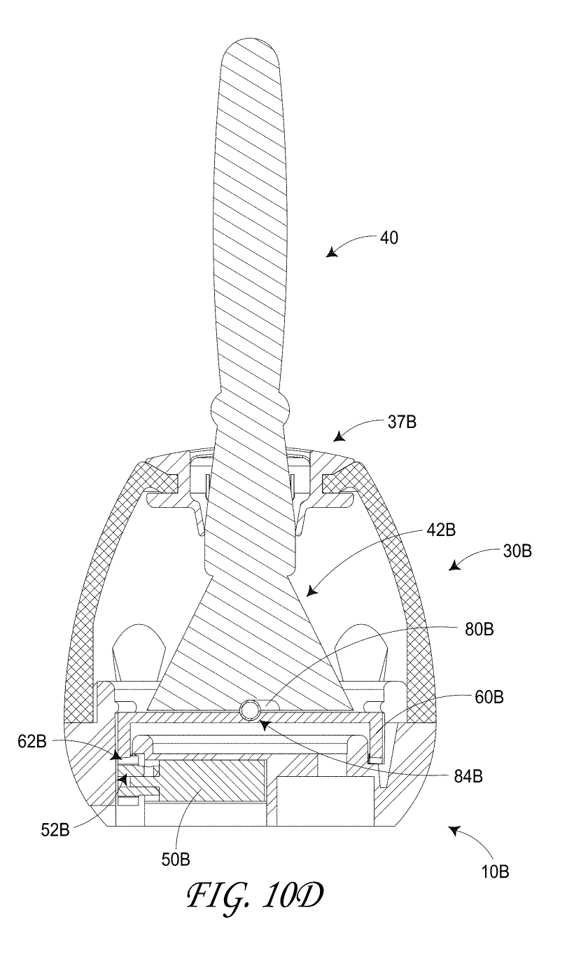

FIGS. 10A-10D show another sterilization unit 100B. The sterilization unit 100B is similar to the sterilization unit 100 in FIGS. 1-8 and includes some of the same components and features disclosed for sterilization unit 100, except as noted below. Thus, the reference numerals used to designate the various components of the sterilization unit 100B are identical to those used for identifying the corresponding components of the sterilization unit 100 in FIGS. 1-8, except that an “B” has been added to the reference numerals, and the description above of the components of the sterilization unit 100 are understood to also apply to corresponding components in the sterilization unit 100B, except as described below.

The sterilization unit 100B differs from the sterilization unit 100 in the construction of the base 10B and the manner in which the light source 80B is moved under the plurality of bristles 42B of the brush 40. The base 10B and the lid 30B optionally have a circular profile symmetrical about a central axis (e.g., axis of symmetry). The sterilization unit 100B has a motor 50B housed in the base 10B that extends along an axis transverse to the central axis (e.g., axis of symmetry) of the base 10B and/or the lid 30B. The motor 50B has an output shaft having a gear 52B. The gear 52B has a plurality of teeth that engage gear teeth 62B of a plate 60B of the base 10B so that the plate 60B rotates about the central axis (e.g., axis of symmetry) of the base 10B. The plate 60B defines the carriage 84A that receives and supports the light source 80A.

When the motor 50B is operated to rotate the geared output shaft 52B, rotation of the geared output shaft 52B rotates the plate 60B about the central axis (e.g., axis of symmetry, vertical axis) of the base 10B. Rotation of the plate 60B causes the carriage 84B to also rotate about the central axis (e.g., axis of symmetry, vertical axis) of the base 10B, thereby rotating the light source 80B under the bristles 42B of the brush 40. Therefore, rotation of the geared output shaft 52B effects rotation of the plate 60B, which in turn effects rotation of the carriage 84B (and the light source 80B disposed thereon) about the central axis (e.g., axis of symmetry) the base 10B. The light sources 80B thereby rotates under the bristles 42B of the brush 40 to separate the bristles 42B from each other to allow debris therebetween to fall out, and simultaneously applies light (e.g., UV light) in between the bristles 42B to sterilize them.

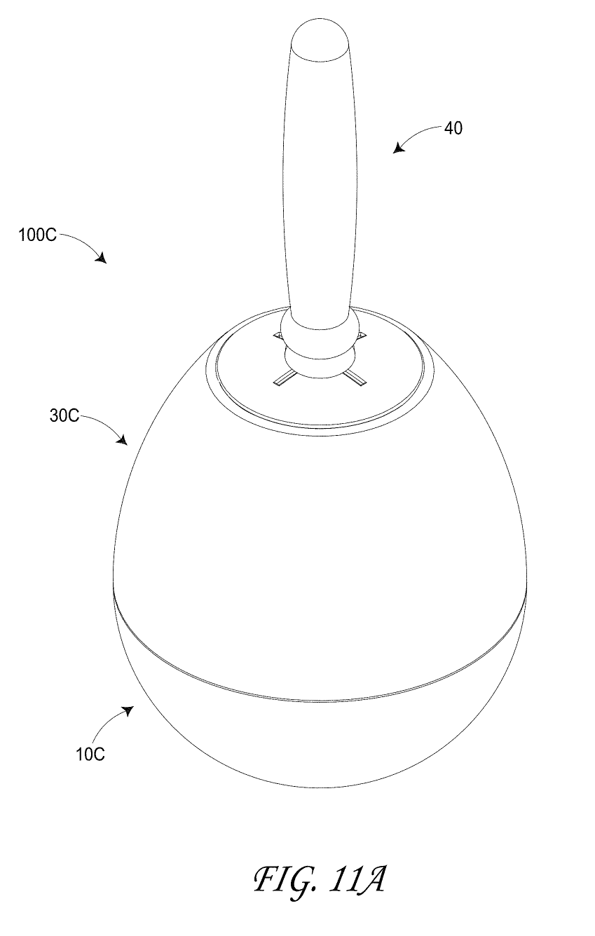

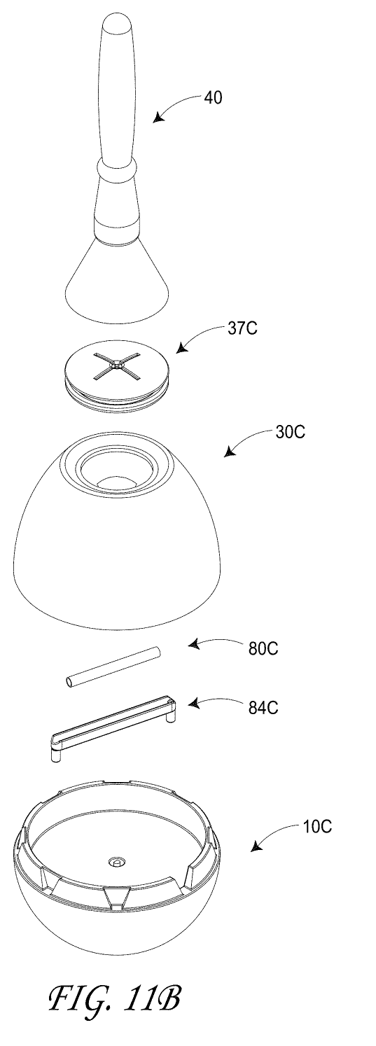

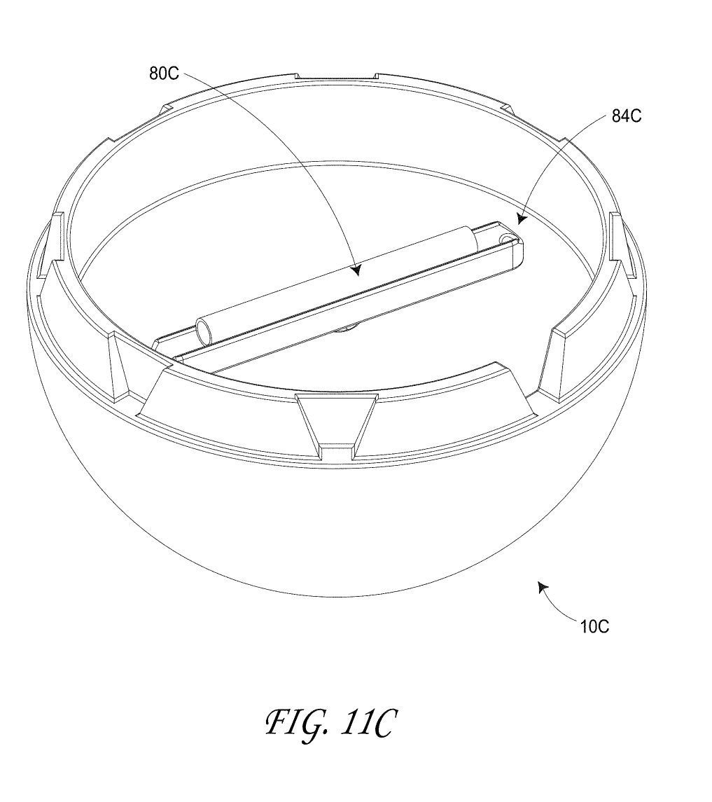

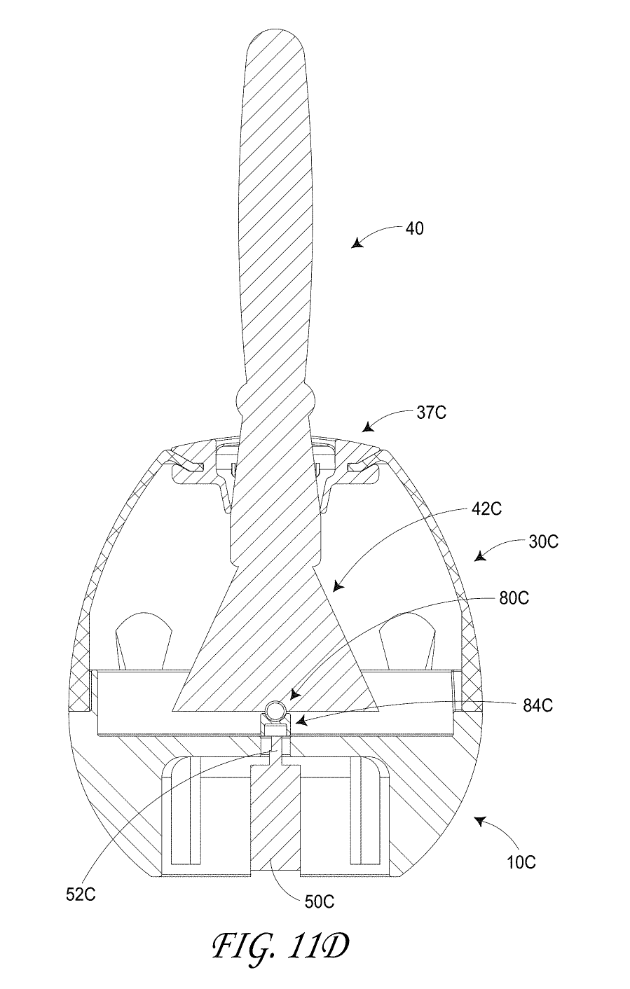

FIGS. 11A-11D show another sterilization unit 100C. The sterilization unit 100C is similar to the sterilization unit 100 in FIGS. 1-8 and includes some of the same components and features disclosed for sterilization unit 100, except as noted below. Thus, the reference numerals used to designate the various components of the sterilization unit 100C are identical to those used for identifying the corresponding components of the sterilization unit 100 in FIGS. 1-8, except that an “C” has been added to the reference numerals, and the description above of the components of the sterilization unit 100 are understood to also apply to corresponding components in the sterilization unit 100C, except as described below.

The sterilization unit 100C differs from the sterilization unit 100 in the construction of the base 10C and the manner in which the light source 80C is moved under the plurality of bristles 42C of the brush 40. The base 10C and the lid 30C optionally have a circular profile symmetrical about a central axis (e.g., axis of symmetry). The sterilization unit 100C has a motor 50C housed in the base 10C that extends substantially coaxial with the central axis (e.g. axis of symmetry) of the base 10C and/or the lid 30C. The motor 50C has an output shaft 52C (e.g., geared output shaft). The output shaft 52C engages a corresponding connection in an underside of the carriage 84C generally located at a center of the length of the carriage 84C. Optionally, the carriage 84C has pegs 85C that extend into a circumferential groove in the base 10C.

When the motor 50C is operated to rotate the output shaft 52C, rotation of the output shaft 52C rotates the carriage 84C about the central axis (e.g., axis of symmetry, vertical axis) of the base 10C, thereby rotating the light source 80C under the bristles 42C of the brush 40. The pegs 85C of the carriage 84C optionally slide along the circumferential groove on the base 10C. Therefore, rotation of the output shaft 52C effects rotation of the carriage 84C (and the light source 80C disposed thereon) about the central axis (e.g., axis of symmetry) the base 10C. The light sources 80C thereby rotates under the bristles 42C of the brush 40 to separate the bristles 42C from each other to allow debris therebetween to fall out, and simultaneously applies light (e.g., UV light) in between the bristles 42C to sterilize them.

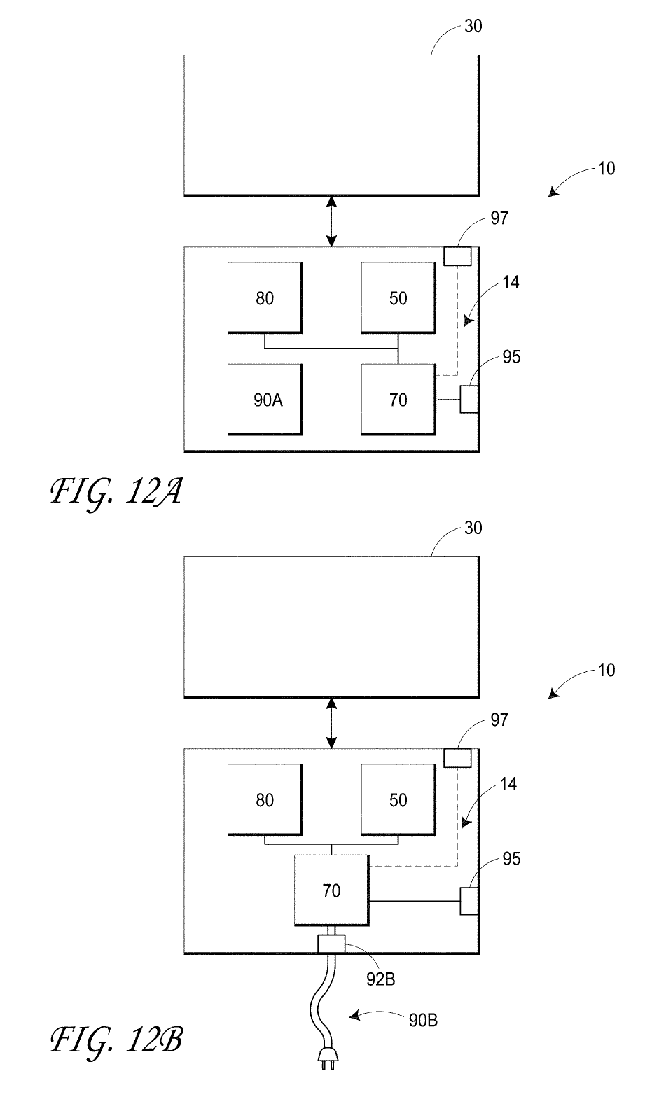

FIG. 12A schematically shows the base 10, in which one or more batteries 90A are disposed in the cavity 14 and connected to the circuitry 70 to thereby provide power to one or more of the circuitry 70, light source 80 and motor 50. FIG. 12B schematically shows the base 10, in which power to one or more of the circuitry 70, motor 50 and light source 80 is provided by an electrical cord 90B that connects to a connector 92B and can optionally be connected to a wall outlet, USB port, etc.

The base 10 can have one or more (e.g., multiple) user interfaces 95 (e.g., button, lever, switch) that communicates with (e.g., is electrically connected to) the circuitry 70. At least one of the user interfaces 95 can be actuated by a user to turn on or turn off power to one or more of the motor 50, light source 80 and circuitry 70. Optionally, at least one of the user interfaces 95 can allow the user to select one or more operating speeds for the motor 50. The base 10 can optionally have a switch 97 (e.g., contact switch, pressure sensitive switch, etc.) that can communicate with (e.g., is electrically connected to) the circuitry 70. The switch 97 can sense when the lid 30 is in the closed position and communicate a signal indicative of said closed position to the circuitry 70. Optionally, the circuitry 70 can operate one or both of the motor 50 and light source 80 upon receipt of both an ON signal from the user interface 95 and the signal from the switch 97 indicating that the lid 30 is in the closed position. Alternatively, the circuitry 70 can operate one or both of the motor 50 and light source 80 upon receipt of the signal from the switch 97 indicating that the lid 30 is in the closed position, without requiring that the user separately actuate the one or more user interfaces 95. In another alternative, the circuitry 70 can operate one or both of the motor 50 and light source 80 upon receipt of the ON signal from the one or more user interfaces 95, without requiring receipt of the signal from the switch 97 indicating the lid 30 is closed.

In operation, the user would move the lid 30 into the open position and insert the brush 40 into the lid 30 so that the handle 46 extends out of the opening 33 and the lid 30 retains the brush 40. The user would then move the lid 30 to the closed position so that the brush head 42 is disposed over the base 10 and so that the one or more light members 82 extend into the brush head 42 and between bristles 42a thereof, as shown in FIG. 7. The user would then operate the unit 100 (e.g., by actuating the one or more user interfaces 95) to cause the motor 50 to move the light source 80 (e.g., rotate the light source, translate the light source fore and aft) under the brush head 42. Advantageously, the one or more light members 82 separate (e.g., open) the bristles 42a from each other as the light source 80 is translated, thereby dislodging debris and dirt from between the bristles, which then fall onto the base 10 (e.g., is collected with the area bounded by the raised lip 17). Additionally, as the light source 80 translates under the brush head 42, illumination of the light (e.g., ultraviolet light) advantageously sterilizes the brush head 42 between the bristles 42a, thereby killing bacteria on the brush head 42. Therefore, the unit 100 advantageously separates the bristles 42a to remove debris and dirt trapped therein, while simultaneously exposing the bristles to light (e.g., ultraviolet light) to kill bacteria on the bristles 42a. The circuitry 70 operates the motor 50 and/or light source 80 for a predetermined period of time to complete the sterilization of the brush 40, after which the unit 100 can optionally provide a signal (e.g., visual, audible) to the user indicating the cleaning process has been completed. Though the description above in connection with FIGS. 12A-12B describes electronics in the base 10, one of skill in the art will recognize that the description can also apply to the electronics in the base 10A, 10B, 10C of the sterilization units 100A, 100B, 100C described above.

While certain embodiments of the inventions have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel”, as well as “generally perpendicular” and “substantially perpendicular”, refer to a value, amount, or characteristic that departs from exactly parallel or exactly perpendicular, respectively, by less than or equal to 15 degrees, less than or equal to 10 degrees, less than or equal to 5 degrees, less than or equal to 3 degrees, less than or equal to 1 degree, or less than or equal to 0.1 degree.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A sterilization unit for a brush, comprising:
a base having a proximal side and a distal side and defining a housing;
a light source movably disposed on the proximal side of the base, the light source configured to move on at least a portion of the proximal side of the base; and
control circuitry disposed in the housing and configured to operate the light source,
wherein movement of the light source simultaneously separates bristles of a brush disposed over the base from each other to thereby remove debris trapped between the bristles and applies light onto the bristles and between the bristles to sterilize them.

2. The unit of claim 1, further comprising an electric motor disposed in the housing and operatively coupled to the light source, the motor operable to actuate movement of the light source.

3. The unit of claim 2, wherein the electric motor effects movement of the light source linearly across at least a portion of the proximal side of the base.

4. The unit of claim 3, wherein the light source is configured to move along a track defined in the proximal side of the base.

5. The unit of claim 2, wherein the electric motor effects rotation of the light source on the proximal side of the base.

6. The unit of claim 1, wherein the light source is an ultraviolet light source.

7. The unit of claim 6, wherein the ultraviolet light source operates at a wavelength of approximately 355 nm.

8. The unit of claim 2, wherein the base comprises an electrical connector configured to connect to a power cord coupleable to a wall outlet to thereby provide power to one or more of the control circuitry, electric motor and light source.

9. The unit of claim 2, wherein the base comprises one or more batteries disposed in the housing and configured to provide power to one or more of the control circuitry, electric motor and light source.

10. The unit of claim 2, further comprising a lid configured to couple to the base, the lid movable between an open position relative to the base where at least a portion of a distal end of the lid is spaced apart from the base to allow access to the proximal side of the base, and a closed position relative to the base where the distal end of the lid is coupled to the base to cover the proximal side of the base, the lid sized to receive at least a portion of a brush therein so that when the lid is in the closed position bristles of the brush are adjacent the proximal side of the base and over the light source so that the light source extends in between one or more of the bristles.

11. The unit of claim 10, further comprising a switch configured to detect when the lid is in the closed position and to communicate a signal to the control circuitry indicative of lid being in the closed position.

12. The unit of claim 11, wherein the control circuitry automatically turns on power to the light source and operates the motor to move the light source under the bristles of the brush upon receiving the signal from the switch.

13. The unit of claim 11, wherein the control circuitry turns on power to the light source and operates the motor to move the light source under the bristles of the brush upon receiving the signal from the switch and an actuation signal from a user interface of the unit.

14. The unit of claim 10, wherein the lid includes an opening at a proximal end configured to receive a handle of the brush therethrough, the lid configured to hold the brush generally upright on the base when the lid is in the closed position.

15. The unit of claim 2, wherein the control circuitry simultaneously turns on power to the light source and operates the motor to move the light source under the bristles of the brush upon receiving an actuation signal from a user interface of the unit.

16. The unit of claim 1, wherein the light source extends along an axis generally perpendicular to an axis of the base.

17. A sterilization unit for a brush, comprising:
a base having a proximal side and a distal side and defining a housing;
at least one ultraviolet light source movably disposed on the proximal side of the base, the at least one ultraviolet light source configured to move on at least a portion of the proximal side of the base;
control circuitry disposed in the housing and configured to operate the at least one ultraviolet light source; and
means for effecting movement of the at least one ultraviolet light source on the proximal side of the base so that the at least one ultraviolet light source moves underneath a brush supported on the base to simultaneously separate bristles of the brush from each other to thereby remove debris trapped between the bristles and also apply ultraviolet light onto the bristles and between the bristles to sterilize them.

18. The unit of claim 17, wherein means for effecting movement of the at least one ultraviolet light source includes means for rotating the at least the one ultraviolet light source.

19. The unit of claim 17, wherein means for effecting movement of the at least one ultraviolet light source includes means for translating the at least one ultraviolet light source.

20. The unit of claim 17, further comprising a lid movably coupled to the base, the lid movable between an open position relative to the base where at least a portion of a distal end of the lid is spaced apart from the base to allow access to the proximal side of the base, and a closed position relative to the base where the distal end of the lid is coupled to the base to cover the proximal side of the base.

21. The unit of claim 17, wherein the control circuitry simultaneously turns on power to the at least one ultraviolet light source and operates movement of at least one ultraviolet light source under the bristles of the brush upon receiving an actuation signal from a user interface of the unit.
